(19) United States
(12) Patent Application Publication
(10) Pub. No.: US 2005/0221325 A1
(43) Pub. Date: Oct. 6, 2005

(54) Detection of secreted polypeptides

(76) Inventor: Greg Thill, Milton, MA (US)

Correspondence Address: FISH & RICHARDSON PC, P.O. BOX 1022, MINNEAPOLIS, MN, 55440-1022, US

(21) Appl. No.: 10/515,356

(22) PCT Filed: May 20, 2003
(86) PCT No.: PCT/US03/15845

§ 371 Date: Apr. 7, 2005

Related U.S. Application Data
(60) Provisional application No. 60/382,795, filed on May 22, 2002.

(51) Int. Cl. C12Q 1/68; C12N 15/08

(52) U.S. Cl. 435/6; 435/451; 530/388.15

(57) ABSTRACT

The invention relates to methods of detecting a secreted polypeptide produced by a cell, as well as to methods for selecting a cell that produces high levels of the secreted polypeptide. Such methods can be used to select a cell producing high levels of a secreted polypeptide encoded by a heterologous nucleic acid that has been introduced into the cell.

### TECHNICAL FIELD

This invention relates to methods of detecting secreted polypeptides, and more particularly to methods for selecting cells that produce high levels of secreted polypeptides.

### BACKGROUND

Secreted proteins generally contain at their amino terminus a signal sequence that directs the ribosomes synthesizing them to the endoplasmic reticulum ER). Protein synthesis is completed on ribosomes attached to the rough ER membrane. Completed polypeptide chains move to the Golgi complex and subsequently are sorted to various destinations. Proteins synthesized and sorted in the secretory pathway include not only those that are secreted from the cell, but also proteins resident in the lumen of the ER, Golgi, and lysosomes, as well as integral proteins in the membranes of these organelles and the plasma membrane.

Most newly made proteins in the ER are incorporated into small transport vesicles that either fuse with the cis-Golgi or with each other to form membrane stacks known as the cis-Golgi reticulum. From the cis-Golgi, certain proteins are retrieved to the ER via a different set of retrograde transport vesicles. In the process called cisternal migration a new cis-Golgi stack with its cargo of luminal protein physically moves from the cis position (nearest the ER) to the trans position (farthest from the ER), successively becoming first a medial-Golgi cisterna and then a trans-Golgi cisterna. During this process, membrane and luminal proteins are constantly retrieved from later to earlier Golgi cisternae by small retrograde transport vesicles. By this process, enzymes and other Golgi resident proteins become localized either in the cis- or medial- or trans-Golgi cisternae.

Proteins destined to be secreted from a cell move by cisternal migration to the trans face of the Golgi and then into a complex network of vesicles termed the trans-Golgi reticulum. From there, a secretory protein is sorted into a secretory vesicle. In all cell types, at least some of the secretory proteins are secreted continuously. These proteins are sorted in the trans-Golgi network into transport vesicles that immediately move to and fuse with the plasma membrane, releasing their contents by exocytosis. In some cells, the secretion of a specific set of proteins is not continuous. These proteins are sorted in the trans-Golgi network into secretory vesicles that are stored inside the cell awaiting a stimulus, e.g., the binding of a hormone to its receptor, for exocytosis.

### SUMMARY

The invention is based, at least in part, on the discovery that a secreted polypeptide can be detected on the surface of a cell that produces the polypeptide. The detection of a secreted polypeptide on the surface of a cell can be used as a marker for cellular productivity of the secreted polypeptide. Accordingly, such methods can be used to select a cell producing high levels of a given secreted polypeptide.

In one aspect the invention features a method of selecting a cell producing a secreted polypeptide, the method including: providing a cell population, wherein the cell population contains a cell containing a heterologous nucleic acid encoding a secreted polypeptide; contacting the cell population with a compound that specifically binds to the secreted polypeptide; detecting the binding of the compound to the secreted polypeptide on the surface of the cell; and selecting the cell based upon the presence or amount of the compound bound to the secreted polypeptide on the surface of the cell.

In another aspect, the invention features a method of generating a cell producing a secreted polypeptide, the method including: introducing into a cell a heterologous nucleic acid encoding a secreted polypeptide; culturing the cell under conditions that allow for synthesis of the secreted polypeptide; contacting the cell with a compound that specifically binds to the secreted polypeptide; detecting expression of the secreted polypeptide by binding of the compound to the secreted polypeptide on the surface of the cell; and selecting the cell by fluorescence activated cell sorting.

A “secreted polypeptide” refers to a protein that is synthesized and sorted in the secretory pathway of a cell and is subsequently released from the cell in a soluble form. A “secreted polypeptide” typically contains an amino terminus signal sequence that is cleaved prior to the release of the polypeptide from the cell. A “secreted polypeptide” does not refer to a species of a protein that exists as an integral membrane protein or that is released from a cell by the cleavage of an integral membrane protein, e.g., wherein the cleavage event releases a soluble extracellular region of the integral membrane protein.

“Selecting a cell” refers to a process of assigning a cell to a given physical location. In the context of the present invention, a cell is assigned a physical location based upon the presence or amount of a compound bound to a secreted polypeptide on the surface of the cell. Cells not having the desired characteristic are typically not assigned to the same physical location as a selected cell. The phrase “selecting a cell” includes, for example, depositing a cell (optionally together with other cells having the same or similar characteristics) in a collection vessel based upon fluorescence properties of the cell as identified by flow cytometry. Other examples of methods for selecting a cell include magnetic separation and panning techniques.

A “heterologous nucleic acid” refers to a nucleotide sequence that has been introduced into a cell by the use of recombinant techniques. Accordingly, a “heterologous nucleic acid” present in a given cell does not naturally occur in the cell (e.g., has no corresponding identical sequence in the genome of the cell) and/or is present in the cell at a location different than that where a corresponding identical sequence naturally exists (e.g., the nucleotide sequence is present in a different location in the genome of the cell or is present in the cell as a construct not integrated in the genome). A “heterologous nucleic acid” does not refer to a nucleotide sequence that is present in a cell as a result of a cell fusion event between two or more cells.

The cell can be, for example, a eukaryotic cell (e.g., a mammalian cell such as a Chinese Hamster Ovary (CHO) cell or a COS cell) or a prokaryotic cell. The cell can be derived from a cell line or can be a primary cell. In one embodiment, the cell is not a transformed cell. In another embodiment, the cell is not a B cell or a cell formed by fusion of a B cell and another cell.

The secreted polypeptide can be an antibody, e.g., a humanized antibody.

The compound can be labeled, e.g., fluorescently labeled. The compound can be an antibody, e.g., a fluorescently labeled antibody.

In one embodiment, the binding of the antibody to the secreted polypeptide on the surface of the cell is detected by flow cytometry. The cell can optionally be selected by fluorescence activated cell sorting.

The cell can be selected together with a plurality of cells in the cell population displaying the compound bound to the secreted polypeptide on the surface of the plurality of cells. The plurality of cells can optionally contain, e.g., at least 1%, 5%, 10%, 20%, 30%, 40%, 50%, or more of the cells in the cell population. The plurality of cells can optionally contain no more than, e.g., at least 1%, 5%, 10%, 20%, 30%, 40%, or 50% of the cells in the cell population.

The cell can be deposited in a vessel containing no cells in addition to the cell.

A method described herein can further include culturing the selected cell to produce a second cell population that produces the secreted polypeptide; contacting the second cell population with the antibody; detecting the binding of the antibody to the secreted polypeptide on the surface of a cell in the second cell population; and selecting the cell in the second cell population by fluorescence activated cell sorting based upon the presence or amount of the antibody bound to the secreted polypeptide on the surface of the cell. In one embodiment, the contacting of the cell population with the antibody is carried out between 4° C. and 10° C., e.g., at about 4° C.

A method described herein can further include culturing the selected cell in culture medium under conditions that allow for secretion of the secreted polypeptide into the culture medium; and purifying the secreted polypeptide from the culture medium.

In another aspect, the invention features a method of determining the presence or amount of a secreted polypeptide produced by a cell, the method including: contacting a cell producing a secreted polypeptide with a compound that specifically binds to the secreted polypeptide, wherein the cell is not a B cell or a cell formed by the fusion of a B cell with another cell; and detecting the binding of the compound to the secreted polypeptide on the surface of the cell, to thereby determine the presence or amount of the secreted polypeptide produced by the cell.

In one embodiment, the cell contains a heterologous nucleic acid encoding the secreted polypeptide.

The cell can be, for example, a eukaryotic cell (e.g., a mammalian cell such as a Chinese Hamster Ovary (CHO) cell or a COS cell) or a prokaryotic cell. The cell can be derived from a cell line or can be a primary cell. In one embodiment, the cell is not a transformed cell.

The secreted polypeptide can be an antibody, e.g., a humanized antibody.

The compound can be labeled, e.g., fluorescently labeled. The compound can be an antibody, e.g., a fluorescently labeled antibody.

In one embodiment, the binding of the antibody to the secreted polypeptide on the surface of the cell is detected by flow cytometry. The cell can optionally be selected by fluorescence activated cell sorting.

In another aspect, the invention features a method of selecting a cell, the method including: providing a cell population containing a plurality of cells genetically engineered to contain a nucleic acid encoding a secreted polypeptide; contacting the cell population with a compound that specifically binds to the secreted polypeptide; and selecting a cell on the surface of which the compound is bound.

The cell can be, for example, a eukaryotic cell (e.g., a mammalian cell such as a Chinese Hamster Ovary (CHO) cell or a COS cell) or a prokaryotic cell. The cell can be derived from a cell line or can be a primary cell. In one embodiment, the cell is not a transformed cell. In another embodiment, the cell is not a B cell or a cell formed by fusion of a B cell and another cell.

The secreted polypeptide can be an antibody, e.g., a humanized antibody.

The compound can be labeled, e.g., fluorescently labeled. The compound can be an antibody, e.g., a fluorescently labeled antibody.

In one embodiment, the binding of the antibody to the secreted polypeptide on the surface of the cell is detected by flow cytometry. The cell can optionally be selected by fluorescence activated cell sorting.

The cell can be selected together with a plurality of cells in the cell population displaying the compound bound to the secreted polypeptide on the surface of the plurality of cells. The plurality of cells can optionally contain, e.g., at least 1%, 5%, 10%, 20%, 30%, 40%, 50%, or more of the cells in the cell population. The plurality of cells can optionally contain no more than, e.g., at least 1%, 5%, 10%, 20%, 30%, 40%, or 50% of the cells in the cell population.

The cell can be deposited in a vessel containing no cells in addition to the cell.

The methods described herein allow for the simple, fast, and direct detection of secreted polypeptides on the cell surface, optionally followed by cell sorting. High-speed cell sorters can sort hundreds of millions of cells with exceptional accuracy, greatly enriching high producer populations.

An advantage of the invention is that, by using the presence of a secreted polypeptide on the surface of a cell to guide cell selection, the methods can greatly facilitate the process of selecting cells producing a given secreted polypeptide. For example, the methods of the invention can reduce the necessity for carrying out extensive labor intensive and costly assays to detect a polypeptide secreted into cell culture media. In addition, the methods of the invention can reduce the number of individual clones that are analyzed during a cell selection process to identify a high producing cell line.

Another advantage of the methods of the invention is that they can be used to comprehensively survey an entire target cell population, since potentially all cells present in a transfected or amplified cell population can be examined for the production of a secreted polypeptide. Methods that rely, for example, on cloning do not provide for the direct detection of relative amounts of a polypeptide secreted by all cells in a population. The methods of the invention permit the direct analysis of a large number of cells and the determination of their relative expression levels for a given secreted polypeptide.

Another advantage of the methods of the invention is that, up to the point of cloning (if cloning is desired), all cells in a target cell population (e.g., cells transfected with a nucleic acid encoding a secreted polypeptide) can be handled in a single batch. As relatively little handling of the cells of target cell population is required, the production of multiple cell lines is therefore facilitated. Immunoassay labor and expense can also be greatly reduced, as initial screening steps can be performed by using a flow cytometer.

Another advantage of the invention is that the methods directly detect the production of a given secreted polypeptide. Methods that instead rely on the detection of a surrogate marker such as a selectable marker or reporter protein can provide good measures of transcription of a nucleic acid encoding a secreted polypeptide, but do not necessarily provide a good measure of secretion of the secreted polypeptide. For example, increased transcription of a nucleic acid does not necessarily correlate with increased translation and secretion of the encoded polypeptide. The methods of the invention allow for the direct selection of a cell that possesses the proper cellular machinery and conditions that lead to high level production of the secreted polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### DESCRIPTION OF DRAWINGS

FIG. 1A is a histogram depicting untransfected CHO cells stained with an RPE labeled goat anti-human antibody.

FIG. 1B is a histogram depicting CHO cells transfected with pXLTBR.9 and stained with an RPE labeled goat anti-human antibody.

FIG. 2A is a histogram depicting pXLTBR.9-transfected CHO cells, following one round of sorting, stained with an RPE labeled goat anti-human antibody.

FIG. 2B is a histogram depicting pXLTBR.9-transfected CHO cells, following two rounds of sorting, stained with an RPE labeled goat anti-human antibody.

FIG. 2C is a histogram depicting pXLTBR.9-transfected CHO cells, following three rounds of sorting, stained with an RPE labeled goat anti-human antibody.

FIG. 3 is a histogram depicting CHO cells transfected with plasmids encoding the AQC2 mAb and stained with an RPE labeled goat anti-human antibody, before cell sorting (left) and after cell sorting (right).

### DETAILED DESCRIPTION

The present invention provides methods for detecting a secreted polypeptide on the surface of a cell that produces the polypeptide. The detection of a secreted polypeptide on the surface of cells can be used to select cells based upon the presence or amount of a given secreted polypeptide produced by the cells.

The screening methods described herein (e.g., screening transfected cell lines for cells that are relatively high producers of a heterologous polypeptide) detect a secreted polypeptide that is transiently associated with the plasma membrane during protein secretion. As such, the secreted polypeptide can be labeled with a compound, e.g., a fluorescent reagent such as a protein-specific antibody. As described in the accompanying Examples, fluorescence intensity of labeled secreted polypeptides on the cell surface was used as the predominant criteria for the selection of clones and resulted in the selection of clones having relatively high specific productivity of the secreted polypeptide.

The methods described herein provide for the simple and direct detection of secreted polypeptides on the cell surface, optionally followed by cell sorting. High-speed cell sorters can sort hundreds of millions of cells with exceptional accuracy, greatly enriching high producer populations, and can deposit one cell per well into plates such as 96 well plates. As described in the accompanying Examples, three rounds of re-iterative sorting followed by single cell seeding was found to result in clones with specific productivities 20 times higher than the unsorted transfected cell population. In addition, the selection of clones by cell sorting followed by methotrexate amplification resulted in a greater than 100 fold enrichment in specific productivity.

Secreted Polypeptides

The invention encompasses methods of identifying and selecting cells expressing a secreted polypeptide. The secreted polypeptide can be a naturally occurring or a non-naturally occurring protein. The secreted polypeptide can be produced naturally by a cell (e.g., without any genetic manipulation of the cell), can be encoded by a heterologous nucleic acid introduced into a cell, or can be produced by a cell following the insertion or activation of sequences that regulate expression of a gene encoding the secreted polypeptide.

Any polypeptide that is secreted from a cell can be used in the methods described herein. For example, secreted polypeptides such as cytokines, lymphokines, and/or growth factors can be produced, and cells producing such polypeptides can be selected according to the methods described herein. Examples of such secreted polypeptides include, but are not limited to, Erythropoietin, Interleukin 1-Alpha, Interleukin 1-Beta, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Lymphotactin, Lymphotoxin Alpha, Monocyte Chemoattractant Protein-1, Monocyte Chemoattractant Protein-2, Monocyte Chemoattractant Protein-3, Megapoietin, Oncostatin M, Steel Factor, Thrombopoietin, Vascular Endothelial Cell Growth Factor, Bone Morphogenetic Proteins, Interleukin-1 Receptor Antagonist, Granulocyte-Colony Stimulating Factor, Leukemia Inhibitory Factor, Granulocyte-Macrophage Colony-Stimulating Factor, Macrophage Colony-Stimulating Factor, Interferon Gamma, Interferon Beta, Fibroblast Growth Factor, Tumor Necrosis Factor Alpha, Tumor Necrosis Factor Beta, Transforming Growth Factor Alpha, Gonadotropin, Nerve Growth Factor, Platelet-Derived Growth Factor, Macrophage Inflammatory Protein 1 Alpha, Macrophage Inflammatory Protein 1 Beta, and Fas Ligand. Cells producing a non-naturally occurring, secreted variant of any the above polypeptides can also be identified and selected according to the methods described herein.

In addition to the secreted polypeptides described above, the methods described herein can also be used to produce a fusion protein that contains all or a portion of a given protein fused to a sequence of amino acids that direct secretion of the fusion protein from a cell. In some cases, such fusion proteins can allow for the secretion of a polypeptide sequence that is not typically secreted from a cell. For example, all or a portion of a protein (e.g., a membrane associated protein such as a receptor or an intracellular protein) can be fused to a portion of an immunoglobulin molecule (e.g., to the hinge region and constant region CH2 and CH3 domains of a human IgG1 heavy chain). In addition, all or a portion of a protein can be fused to a heterologous signal sequence. Examples of proteins that can be fused to a sequence that directs secretion of the fusion protein include, but are not limited to, receptors (e.g., Lymphotoxin-Beta receptor), including receptors for any of the naturally occurring secreted polypeptides described herein. In preparing a nucleic acid encoding a fusion protein, a naturally occurring transmembrane segment of a cell surface receptor can be removed to facilitate secretion of the fusion protein encoded by the nucleic acid.

The secreted polypeptide can be an antibody or an antigen-binding fragment of an antibody. The antibody can be directed against an antigen, e.g., a protein antigen such as a soluble polypeptide or a cell surface receptor. For example, the antibody can be directed against a cell surface receptor involved in immune cell activation (e.g., CD3, CD4, CD8, CD40, or an integrin such as alpha 1 beta 1 integrin), a disease-associated antigen (e.g., a cancer-associated antigen such as HER2 or prostate specific membrane antigen), or an antigen produced by a pathogen (e.g., a viral or bacterial antigen). The particular epitope bound by the antibody can be formed by amino acids, carbohydrates (e.g., sugars), inorganic moieties (e.g., phosphates), or combinations thereof. Such epitopes can be found in N- or O-linked glycoproteins, proteoglycans, and phosphorylated proteins.

The term “antibody” refers to an immunoglobulin molecule or an antigen-binding portion thereof. As used herein, the term “antibody” refers to a protein containing at least one, for example two, heavy chain variable regions (“VH”), and at least one, for example two, light chain variable regions (“VL”). The VH and VL regions can be further subdivided into regions of hypervariability, termed “complementarity determining regions” (“CDR”), interspersed with regions that are more conserved, termed “framework regions” (FR). The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region contains three domains, CH1, CH2, and CH3. The light chain constant region contains one domain, CL. The variable region of the heavy and light chains contains a binding domain that interacts with an antigen.

The secreted polypeptide can be a fully human antibody (e.g., an antibody made in a mouse genetically engineered to produce an antibody from a human immunoglobulin sequence), or a non-human antibody, e.g., a rodent (mouse or rat), goat, or primate (e.g., monkey) antibody.

An antibody can be one in which the variable region, or a portion thereof, e.g., the CDRs, are generated in a non-human organism, e.g., a rat or mouse. Chimeric, CDR-grafted, or humanized antibodies can be used as a secreted polypeptide in the methods described herein.

An antibody can be humanized by methods known in the art. For example, humanized antibodies can be generated by replacing sequences of the Fv variable region which are not directly involved in antigen binding with equivalent sequences from human Fv variable regions. General methods for generating humanized antibodies are described by, e.g., Morrison (1985) Science 229:1202-1207.

Nucleic Acids Encoding Secreted Polypeptides

The invention encompasses methods of identifying and selecting cells expressing a secreted polypeptide. In some embodiments, the secreted polypeptide is encoded by a heterologous nucleic acid introduced into a cell or is produced by a cell following the insertion or activation of sequences that regulate expression of a gene encoding the secreted polypeptide.

Any method for introducing a nucleic acid into a cell can be used to produce a secreted polypeptide encoded by a heterologous nucleic acid. The nucleic acid can be naked or associated or complexed with a delivery vehicle. For a description of the use of naked DNA, see e.g., U.S. Pat. No. 5,693,622.

A nucleic acid can be introduced into a cell by a transfection method such as calcium phosphate transfection, transfection using DEAE-Dextran, transfection by electroporation, or transfection using cationic lipid reagents. Suitable transfection methods are described in, e.g., Current Protocols in Molecular Biology (1999) John Wiley & Sons, Inc.

Nucleic acids can be delivered to a cell using delivery vehicles, such as lipids, depot systems, hydrogel networks, particulates, liposomes, ISCOMS, microspheres or nanospheres, microcapsules, microparticles, gold particles, virus-like particles, nanoparticles, polymers, condensing agents, polysaccharides, polyamino acids, dendrimers, saponins, adsorption enhancing materials, colloidal suspensions, dispersions, powders, or fatty acids.

Viral particles can also be used, e.g., retroviruses, adenovirus, adeno-associated virus, pox viruses, SV40 virus, alpha virus, lentivirus, or herpes viruses, to introduce the heterologous nucleic acid into a cell.

Microparticles or nanoparticles can be used as vehicles for delivering nucleic acids into a cell. Microparticles can contain macromolecules embedded in a polymeric matrix or enclosed in a shell of polymer. Microparticles act to maintain the integrity of the macromolecule, e.g., by maintaining the DNA in a nondegraded state.

Nucleic acid constructs encoding a secreted polypeptide can optionally include a nucleotide sequence encoding a selectable marker or a reporter protein. In some cases, a nucleotide sequence encoding a selectable marker or a reporter protein is contained in a second nucleic acid construct that is co-introduced into a cell with the nucleic acid construct encoding the secreted polypeptide. The selectable marker or reporter protein can provide an additional mechanism, in addition to the screening methods described herein, for identifying cells containing a nucleic acid encoding the secreted polypeptide. Selectable markers include, for example, proteins that confer resistance to neomycin, kanamycin, hygromycin, or methotrexate. Reporter proteins include, for example, beta galactosidase, luciferase, and fluorescent proteins such as green fluorescent protein. The detection and selection methods described herein can be carried out in the presence or in the absence of a selectable marker or a reporter protein.

Selection of a Cell Producing a Secreted Polypeptide

A cell producing a secreted polypeptide can be identified by contacting the cell with a compound that specifically binds to the secreted polypeptide and detecting the binding of the compound to the secreted polypeptide on the surface of the cell. In addition, the cell can be selected from other cells based upon the presence or amount of the compound bound to the secreted polypeptide on the surface of the cell. “Selecting” a cell includes isolating a single cell into a vessel containing only that cell (e.g., single cell sorting for the cloning a cell), as well as isolating the cell together with a plurality of cells based upon the cells' similar characteristics with respect to the binding of the compound to the secreted polypeptide on the surface of the cells.

A cell can be selected from other cells in a cell population by the use of flow cytometry and cell sorting techniques. In flow cytometry, measurements of cells are made as the cells flow in single file in a fluid stream past optical and/or electronic sensors. Flow cytometers typically use lasers as light sources and measure light scattered by cells, which provides information about their size and internal structure, and fluorescence in several spectral regions emitted by dyes or labeled probes or reagents that bind specifically and stoichiometrically to cellular constituents such as antigens. Flow sorting allows cells with preselected characteristics to be diverted from the stream and collected for further analysis. The optics of a flow cytometer are similar to those of a fluorescence microscope. Flow cytometry and cell sorting are described in detail in, e.g., Darzynkiewicz et al. (2000) Flow Cytometry, 3rd Edition, San Diego, Academic Press, 2000; and Givan (2001) Flow Cytometry: First Principles, 2nd Edition, New York, Wiley-Liss.

For flow cytometry and cell sorting, the compound that specifically binds to the secreted polypeptide can be a protein such as an antibody. The antibody can have a label, e.g., a fluorescent label, attached to it. Alternatively, a secondary compound (e.g., a secondary antibody) can be used that specifically binds to a primary antibody, wherein the secondary compound either contains a label or is bound by another compound that contains a label. For example, an antibody that binds to the secreted polypeptide can be labeled, e.g., biotinylated, and then contacted to the secreted polypeptide. The antibody-secreted polypeptide complex can be detected, e.g., with avidin coupled to a fluorescent label.

Cells can be subjected to one ore more rounds of sorting according to the methods described herein. Multiple rounds of sorting can be used to enrich for cells producing particularly high levels of the secreted polypeptide. Cells can be cultured between rounds of cell sorting, or cells can be re-sorted without any culture period between the sorting procedures. Cells can optionally be sorted based upon their expression of two or more different secreted polypeptide or a secreted polypeptide and a reporter protein. Additional parameters including but not limited to cell size, cell viability, or the expression of other cell surface markers can also be used in the sorting procedure.

In addition to flow cytometry and cell sorting, cells can be selected by a variety of techniques that allow for the selection of cells having a compound specifically bound to a secreted polypeptide on the surface of the cell. Examples of such selection methods include magnetic separation techniques (e.g., using magnetically labeled compounds such as antibodies that are specifically attracted to magnetic beads) or panning techniques. For a description of magnetic separation and panning techniques, see, e.g., Murphy et al. (1992) J. Cell Sci. 1992 102:789-98.

In some emobidements, the methods described herein entail detecting the binding of the compound to the secreted polypeptide on the surface of the cell without adding a substance to the cell that encapsulates the cell (e.g., forms a matrix around the cell) and/or immobilizes the secreted polypeptide near the cell. For example, buffers used for contacting a compound to a cell and washing unbound compound from the cell can be standard buffers used for flow cytometry and cell sorting (e.g., phosphate buffered saline, optionally including fetal calf serum).

The cells to be detected and/or selected according to the methods described herein can be maintained in a temperature range of approximately 4° C.-10° C. (e.g., about 4° C.) while the cells are contacted with a compound that binds to the secreted polypeptide as well as during associated incubation and cell washing periods. The handling of the cells at a relatively low temperature may facilitate their retention of the secreted polypeptide on the surface of the cell and the subsequent detection of the cell by the specific binding of a compound.

Detection and Purification of Secreted Polypeptides

In addition to the cell-associated screening methods described herein, a secreted polypeptide can be detected in tissue culture media following the secretion of the polypeptide from a given cell. Such methods can be used to quantitate the amount of secreted polypeptide produced by a given cell. For example, an aliquot of tissue culture medium from a cell culture containing a cell sorted as described herein can be used to determine the amount of a given secreted polypeptide contained therein. Such measurements can be used to verify that a cell selected according to a method described herein is secreting the secreted polypeptide or is secreting a defined concentration of the secreted polypeptide. Methods for the detection of the secreted polypeptide include, but are not limited to, enzyme linked immunosorbent assay (ELISA), immunoprecipitation, immunofluorescence, enzyme immunoassay (EIA), radioimmunoassay (RIA), and Western blot analysis. Biological assays can also be carried out to determine the bioactivity of the secreted polypeptide. The nature of the biological assay can vary according to the biological function of the secreted polypeptide.

The secreted polypeptide can optionally be purified from tissue culture medium containing cells that produce the secreted polypeptide. Purification can be accomplished by contacting the culture medium with an affinity agent, e.g., an antibody, that specifically binds to the secreted polypeptide. The secreted polypeptide can optionally be purified to homogeneity.

This invention is further illustrated by the following example that should not be construed as limiting the scope of the invention.

### EXAMPLES

### Example 1

### Direct, Product-Specific Staining of Secreted Recombinant Proteins at the Plasma Membrane with Fluorescently Labeled Antibodies

CHO cells were transfected with the plasmid vectors pAND162 and pAND160, respectively encoding the light and heavy chains of a humanized monoclonal antibody to alpha 1 beta 1 integrin (AQC2 mAb). Plasmid pAND162 encodes a neomycin resistance selectable marker, and plasmid pAND160 encodes wild type DHFR. Both plasmids use the CMV intermediate-early promoter, which extends from a restriction site approximately 500 bp upstream of the TATA box to a polylinker near the initiation codon of the native CMV intermediate early gene. The promoter region includes splice donor and acceptor sites in the 5′ untranslated region. The polyadenylation site is derived from human growth hormone variant sequence.

DHFR deficient DG44 CHO host cells were maintained as spinner cultures in serum free medium containing nucleosides. Transfections were carried out by electroporation. Transfected cell lines were grown in alpha minus MEM supplemented with 10% dialyzed fetal bovine serum (FBS) (Hyclone, Logan, Utah) and 2 mM Glutamine (Life Technologies, Grand Island, N.Y.). Following electroporation, the cells were cultured in 6-well tissue culture dishes (Becton Dickinson, Franklin Lakes, N.J.). Three days post-infection, 400 μg/ml G418 (Geneticin, Life Technologies, Grand Island, N.Y.) was added to the medium containing alpha minus MEM supplemented with 10% dialyzed FBS and 2 mM glutamine. Once cells had reached about 80% confluence, the wells were pooled and sorted.

CHO cells transfected with the pAND162 and pAND160 plasmids encoding the humanized AQC2 antibody were labeled with a fluorescently labeled anti-human antibody. Staining of the cells was then viewed using laser confocal microscopy. The cells were kept on ice until confocal analysis. Fluorescent and differential interference contrast photomicrographs were acquired on a Leica TCS SP confocal microscope equipped with a red laser diode and Leica confocal software V2.00 build 0368 (Leica Microsystems, Heidelberg GmbH, Germany). Photomicrographs were taken of cells observed through a 40× oil immersion objective. Intense staining of the plasma membrane of the transfected cells with the anti-human antibody was detected. No staining of the plasma membrane of the untransfected DG44 CHO host cells was detected. As the detectors used in flow cytometry are more sensitive than those used in laser confocal microscopy, flow cytometry was also used to examine populations of transfected CHO cells that had been labeled with fluorescent reagents directed against the secreted humanized AQC2 antibody (see Example 3).

### Example 2

### Generation of High Producing Recombinant CHO Cell Lines in the Absence of Methotrexate

Plasmid pXLTBR.9 contains a nucleotide sequence encoding wild type DHFR as well as a nucleotide sequence encoding the lymphotoxin-beta receptor fused to human IgG domains, C_{H}2 and C_{H}3 (LTbetaR-Ig) (Browning et al. (1995) J. Immunol. 154:33). pXLTBR.9 uses the CMV intermediate-early promoter, as described in Example 1 for pAND162 and pAND160.

DG44 CHO cells were transfected with pXLTBR.9 by electroporation according to the methods described in Example 1. The pXLTBR.9 transfected cell lines were grown in HYQPF-CHO (Hyclone Laboratories, Logan, Utah), a serum-free medium, or Serum-Free alpha plus MEM medium (alpha plus MEMSF), an enriched alpha plus MEM without FBS.

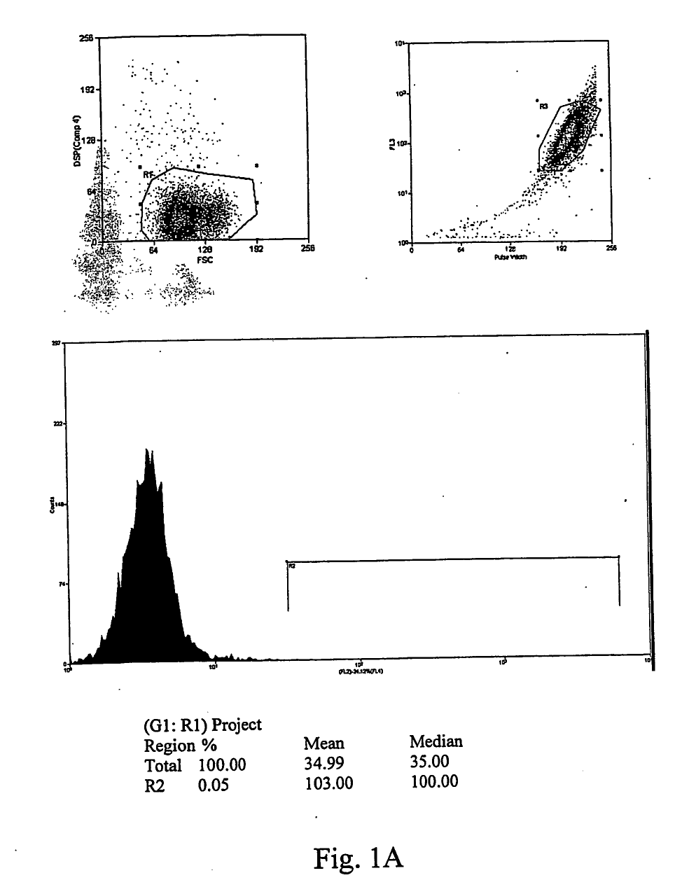

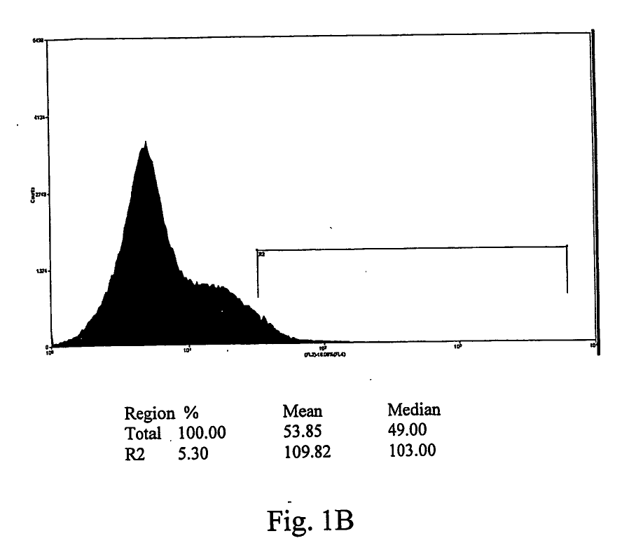

After selection and outgrowth of the pXLTBR.9 transfectants for more than 14 days, the cells were pooled and labeled with an RPE labeled F(ab′)₂ fragment of an goat anti-human IgG molecule at 4° C. These cells, along with stained negative control CHO cells, were subjected to analytical flow cytometry prior to a preparative sort. FIG. 1A displays a histogram of negative control, untransfected CHO cells. The FL-2 histogram was derived from the combination of the live cell gate (based on PI exclusion, top left), and the double discrimination gate (pulse width vs FSC, to exclude doublets, top right). R2 represents the sorting gate. FIG. 1B displays a histogram of CHO cells transfected with pXLTBR.9. The sort gate R2 was set to collect the brightest 5% of R-PE positive cells for all three reiterative sorts. The transfected cells (FIG. 1B) contained populations of cells from which the fluorescence intensity greatly exceeded that of the negative control (FIG. 1A). For preparative sorting of the transfected cells, a gate was set that encompassed cells within the top 5% of the fluorescence intensity of the cell population. The gated cells were sorted and their cell number was expanded by culture under selective conditions and the process was repeated two more times.

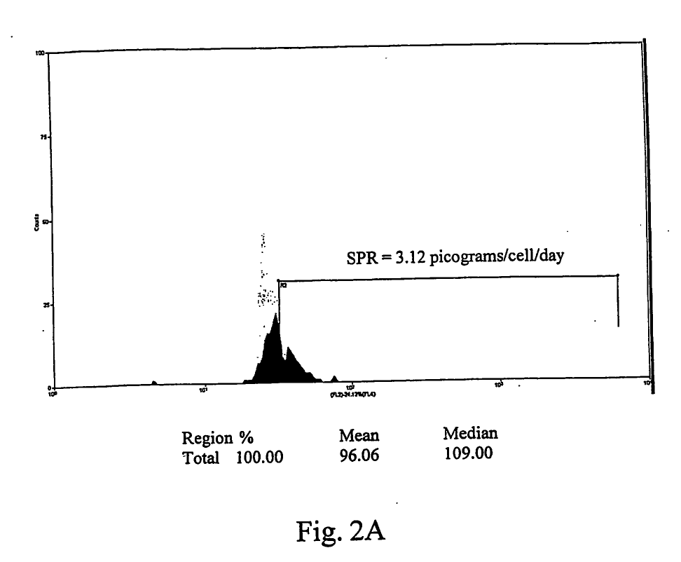

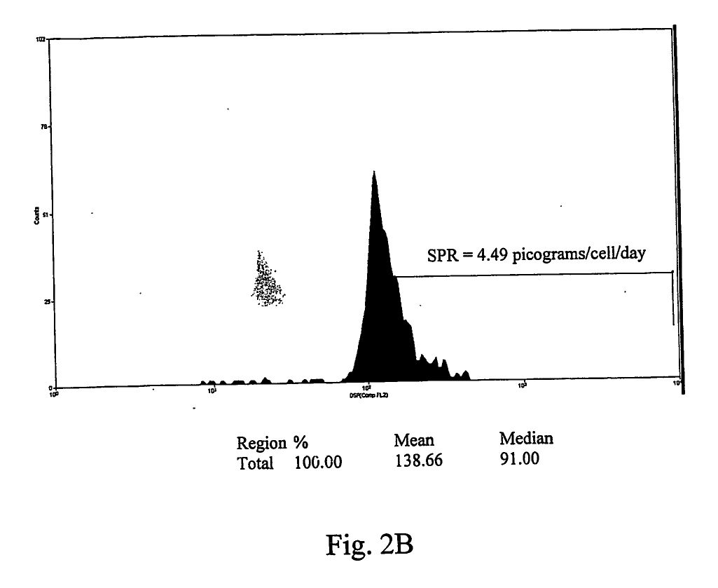

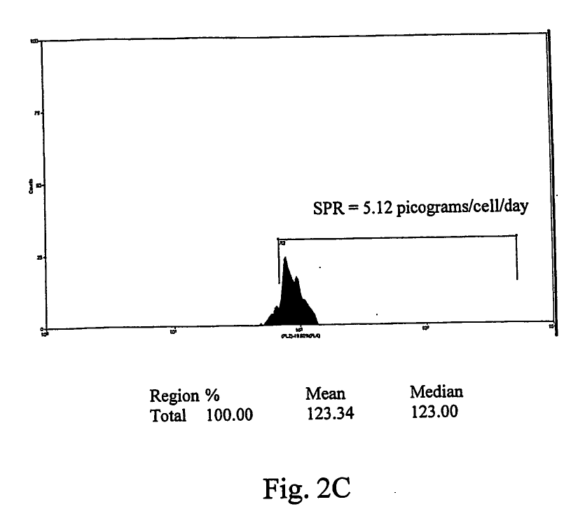

For LTbetaR-Ig producing cell lines, an analytical scan was performed post-sorting to evaluate the quality of the sort. The analytical scan as well as the experimentally determined specific productivity rates (SPR) of LTbetaR-Ig in the pools are displayed in FIGS. 2A-2C. Unsorted transfected cells had a SPR of approximately 0.5 pg/cell/day (pcd). FIG. 2A, an analytical scan of a sample of LTbetaR-Ig-producing cells collected after a first sort, shows that the sort resulted in a population with an increased mean fluorescence intensity and a corresponding increase in specific productivity (the SPR values were determined after expansion of the cells in culture). FIG. 2B, an analytical scan of a sample of LTbetaR-Ig-producing cells collected after a second sort, demonstrates a progressive increases in both fluorescence intensity and specific productivity after reiterative sorting. FIG. 2C is an analytical scan of a sample of LTbetaR-Ig-producing cells collected after a third sort. The three rounds of sorting of the cells improved the SPR average of the pools by approximately ten-fold to 5.1 pcd (Table 1).

TABLE 1
Specific Productivity Rate of Unsorted And Consecutively
Sorted CHO Pools of LTbetaR-Ig Expressing Cells Demonstrates
That Specific Productivity Increases with Re-Iterative Sorting

| | Average SPR |
|---|---|
| LTbetaR-Ig pool | (pg cell⁻¹ day⁻¹) ± S.D. |
| Unsorted | 0.5 ± 0.0 |
| First sort | 3.1 ± 0.1 |
| Second sort 2 | 4.5 ± 0.3 |
| Third sort 3 | 5.1 ± 0.7 |

For the SPR assay results depicted in Table 1, 2×10⁵ cells were cultured in a 9.6 cm tissue culture dish in 2 ml of serum-containing media. The cells and supernatant were harvested after three days in culture. The SPR assay was conducted in triplicate for each sample.

During the third sort, clones were isolated directly from the cytometer into 96 well plates. Several weeks later, assayed clones demonstrating a range of specific productivity between 4 and 11.5 pcd were maintained (Table 2). The most productive clone demonstrated a twenty-three-fold enrichment in specific productivity, without the need for methotrexate amplification. Fifty clones assayed by ELISA during this enrichment process. The timeline was approximately nine weeks from transfection to identification of the best clone.

TABLE 2
Specific Productivity Rates of LTbetaR-Ig Expressing CHO Clones
Isolated From Pools Subjected to Three Re-Iterative FACS Sorts

| LTbetaR-Ig clone | Average SPR (pg cell⁻¹ day⁻¹) |
|---|---|
| 1 | 3.6 ± 0.1 |
| 3 | 5.6 ± 0.6 |
| 5 | 11.5 ± 0.2 |
| 8 | 7.5 ± 0.1 |
| 21 | 9.9 ± 1.4 |
| 22 | 7.5 ± 0.7 |
| 24 | 9.3 ± 0.8 |
| 25 | 10.5 ± 1.1 |

In the experiments depicted in Table 2, triplicate three-day SPR assays were conducted in serum-containing media for each sample.

In the experiments described herein, cells were harvested before sorting by Accutase treatment (Innovative Cell Technologies, La Jolla, Calif.) and then maintained at 0-4° C. for all subsequent handling. The cells were passed through a 70 μm nylon mesh (Becton Dickinson Labware, Franklin Lakes, N.J.), washed twice with cold phosphate buffered saline (PBS) (Life Technologies, Grand Island, N.Y.), and then counted and assessed for viability. The cells were pelleted again by centrifugation for 5 minutes at 1,000 RPM at 4° C., and resuspended in cold DMEM/BSA containing fluorescently labeled antibody.

A minimum of 1×10⁷ cells were stained for the detection of plasma membrane surface LTbetaR-Ig (or humanized AQC2 mAb in the Example 3) with R-phycoerythrin (RPE) conjugated goat F(ab′)2 anti-human IgG (Jackson Immunoresearch, West Grove, Pa.), at a concentration of 0.2-1 ug antibody per 1×10⁵ cells in Dulbecco's Minimal Essential Media (DMEM) (Life Technologies, Grand Island, N.Y.), supplemented with 2% Bovine Serum Albumin (13SA) (Sigma Chemical Co, St. Louis, Mo.). After a 15 minute incubation on ice, the cells were washed twice with cold PBS, and resuspended in PBS plus 2 ug/ml propidium iodide (PI) (Molecular probes, Eugene, Oreg.). Approximately 5×10⁵ cells were removed for pre-analysis by a FACScan cytometer before sorting. Untransfected DG44 CHO cells were used as a negative control.

Analytical flow cytometry scans were performed on a FACScan flow cytometer (Becton Dickinson, San Jose, Calif.) equipped with Cellquest v3.0 software and an air-cooled argon laser emitting at 488 nm. The PE emission was detected on Fl-2 and the PI emission was detected on Fl-3 using a 585 nm band pass filter.

High speed cell sorts were performed on a Moflo flow cytometer (Cytomation, Fort Collins, Colo.), equipped with Summit 3.0 software and an argon laser emitting at 488 nm for fluorescence excitation. The PE emission was detected on Fl-2, using a 670/40 nm band pass filter, and the PI emission was detected on Fl-4, using a 670/40 band pass filter. Compensation of PE/PI emission spectrum overlap was accomplished using Cytomation's DSP (Digital Signal Processing) board in Summit. Dead cells were excluded in a FSC vs. Fl-4 dot plot and doublets were excluded in a PSC Width vs. Area dot plot. PE-labeled signal gating was done on a live cell gated Fl-2 histogram. The sorting gate was the combination of the live cell gate, the double discrimination gate, and the histogram gate on Fl-2.

LTbetaR-Ig titers were determined from tissue culture supernatant by ELISA. Assay plates were coated with an LTbetaR-Ig antibody and bound LTbetaR-Ig was detected by anti-human IgG horseradish peroxidase (HRP) conjugate (Jackson Immunoresearch Laboratories, Inc., West Grove, Pa.). The concentration of LTbetaR-Ig was determined by linear regression analysis of the standards.

For each population or clone, 1×10⁵ cells were seeded per well of a 6-well tissue culture plate (Corning Inc., Corning, N.Y.), in 2 ml of growth media. Assays were performed in triplicate. The cells were allowed to grow for 3 days, conditioned media was harvested for analysis, and the cells removed by Accutase and counted. Specific LTbetaR-Ig (or AQC2 antibody, as in Example 3) titers were quantitatively determined from media samples by ELISA. The SPR measured in picograms of specific protein per cell per day (pg cell⁻¹day⁻¹), is a function of both growth rate and productivity, as represented by the following equations:

$$SPR = \frac{Total protein mass}{Integral cell area ( ICA )} = qP$$

$$ICA = \frac{( final cell number - initial cell number ) \times days in culture}{LN ( final cell number / initial cell number )}$$

### Example 3

### Generation of Methotrexate Amplified Recombinant CHO Cell Lines

The light and heavy chains of a humanized antibody to alpha 1 beta 1 integrin (AQC2 mAb) were expressed in CHO cells (as described in Example 1) from separate plasmids pAND162 and pAND160. After transfection and expansion under DFR and G418 selection, the entire transfected cell population, having a specific productivity of 0.3 pcd, was labeled with a fluorescent F(ab′)₂ fragment of goat anti-human IgG, and the top 2-5% expressing cells as measured by fluorescence intensity were collected by cell sorting. After approximately one week of expansion, sorted cells were subjected to a second sort. The cells were expanded again, then deposited at one cell per well into 96 well plates during a third sort. As in the case of LTbetaR-Ig (Example 2), sorting resulted in a steady increase in the fluorescence intensity of the labeled cells as well as the measured specific productivity of both pools and clones.

For the quantitation of AQC2 mAb by ELISA, assay plates were coated with an AQC2 specific antigen fusion protein. Bound AQC2 mAb was detected with donkey anti-human IgG (H+ L) horseradish peroxidase conjugate (Jackson ImmunoResearch, West Grove, Pa.).

Approximately 117 clones were expanded into 24 well plates and screened for antibody titer. The top expressing clones were further analyzed in a SPR assay. G418 was removed from the highest ten expressing clones, which were then further amplified in media containing either 100 nM or 250 nM methotrexate. Amplified pools were screened for antibody titer. Populations exhibiting a qP equal to or greater than 13.5 pcd were subjected to high speed cell sorting and autocloning of the upper 2% expressing population to 96 well plates. Two of the top antibody-producing clones, clone 5A and 11B had unamplified qPs of 3.3 and 8.0 pcd, respectively (Table 3). When clone 5A was amplified in the presence of 250 nM methotrexate a pool of specific productivity of 16.6 pcd was generated. After fluorescent activated cell sorting and cloning, the best producers from 52 clones assayed exhibited qPs of 41.0 and 32.3 pcd. Similarly, for clone 11B, cells amplified only in 100 nM methotrexate had a specific productivity of 18 pcd and produced clones of up to 32.5 pcd out of approximately 126 clones screened (Table 3).

TABLE 3
Specific Productivity Rates of AQC2 mAb Expressing CHO Cells
Isolated from Pools Subjected to Three Re-Iterative
FACS Sorts Before and After Methotrexate (MTX) Amplification

| | | | SPR | # clones screened |
|---|---|---|---|---|
| | | | (pg cell⁻¹ | from amplified |
| | | nM MTX | day⁻¹) | pool |
| Unamplified | 5A | — | 3.3 | |
| parent clone | | | | |
| Amplified pool | 5AB | 250 | 16.6 | 52 |
| Amplified | 5AB-17 | 250 | 41.0 | |
| subclone | 5AB-52 | 250 | 32.3 | |
| Unamplified | 11B | — | 8.0 | |
| parent clone | | | | |
| Amplified pool | 11BB | 250 | 13.5 | |
| Amplified | 11BB-46 | 250 | 25.4 | 121 |
| subclone | 11BB-67 | 250 | 27.3 | |
| | 11BB-68 | 250 | 19.9 | |
| | 11BB-83 | 250 | 26.9 | |
| Amplified pool | 11BA | 100 | 17.9 | |
| Amplified | 11BA-1 | 100 | 18.4 | 126 |
| subclone | 11BA-30 | 100 | 19.9 | |
| | 11BA-41 | 100 | 18.2 | |
| | 11BA-47 | 100 | 26.6 | |
| | 11BA-50 | 100 | 26.1 | |
| | 11BA-118 | 100 | 28.1 | |
| | 11BA-100 | 100 | 32.5 | |

For the experiments depicted in Table 2, triplicate three-day SPR assays were conducted in serum-containing media for each sample.

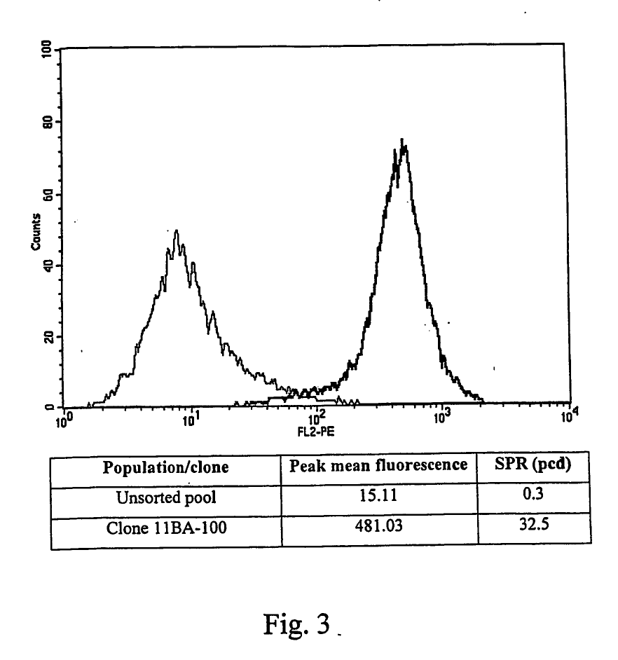

FIG. 3 depicts an analytical scan of unsorted CHO cell transfected with plasmids encoding the AQC2 mAb (left) versus amplified clone 11BA-100 (right), demonstrating an increase in both mean fluorescence intensity and specific productivity after methotrexate amplification, sorting, and cloning. The FL-2 histograms were derived by analysis of the PE signal within the live cell gate. FIG. 3 shows a thirty-two-fold increase in the mean fluorescence intensity of the top 100 nM methotrexate amplified clone 11BA-100, compared to the initial pool of transfectants (which also correlates to the high level product secretion). The 250 nM methotrexate amplified pool had a qP of 13.5 pcd and produced clones of up to 27 pcd in a similar size screen (Table 3). The increase in fluorescence intensity correlates to the significant increase in protein secretion. As was seen in the case of the LTbetaR-Ig fusion protein (Example 2), fluorescence intensity was a useful surrogate marker for specific cellular productivity of a secreted protein.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of selecting a cell producing a secreted polypeptide, the method comprising:
providing a cell population, wherein the cell population comprises a cell comprising a heterologous nucleic acid encoding a secreted polypeptide;
contacting the cell population with a compound that specifically binds to the secreted polypeptide;
detecting the binding of the compound to the secreted polypeptide on the surface of the cell; and
selecting the cell based upon the presence or amount of the compound bound to the secreted polypeptide on the surface of the cell.

2. The method of claim 1, wherein the cell is not a transformed cell.

3. The method of claim 1, wherein the cell is a eukaryotic cell.

4. The method of claim 3, wherein the cell is a mammalian cell.

5. The method of claim 4, wherein the cell is a Chinese Hamster Ovary (CHO) cell.

6. The method of claim 1, wherein the cell is not a B cell or a cell formed by fusion of a B cell and another cell.

7. The method of claim 1, wherein the secreted polypeptide is an antibody.

8. The method of claim 7, wherein the antibody is a humanized antibody.

9. The method of claim 1, wherein the compound is labeled.

10. The method of claim 9, wherein the compound is fluorescently labeled.

11. The method of claim 1, wherein the compound is an antibody.

12. The method of claim 11, wherein the binding of the antibody to the secreted polypeptide on the surface of the cell is detected by flow cytometry.

13. The method of claim 12, wherein the cell is selected by fluorescence activated cell sorting.

14. The method of claim 13, wherein the cell is selected together with a plurality of cells in the cell population displaying the compound bound to the secreted polypeptide on the surface of the plurality of cells.

15. The method of claim 14, wherein the plurality of cells comprises at least 1% of the cell population.

16. The method of claim 15, wherein the plurality of cells comprises at least 5% of the cell population.

17. The method of claim 13, wherein the cell is deposited in a vessel containing no cells in addition to the cell.

18. The method of claim 13, further comprising:
culturing the selected cell to produce a second cell population that produces the secreted polypeptide;
contacting the second cell population with the antibody;
detecting the binding of the antibody to the secreted polypeptide on the surface of a cell in the second cell population; and
selecting the cell in the second cell population by fluorescence activated cell sorting based upon the presence or amount of the antibody bound to the secreted polypeptide on the surface of the cell.

19. The method of claim 13, wherein the contacting of the cell population with the antibody is carried out between 4° C. and 10° C.

20. The method of claim 19, wherein the contacting of the cell population with the antibody is carried out at about 4° C.

21. The method of claim 1, further comprising:
culturing the selected cell in culture medium under conditions that allow for secretion of the secreted polypeptide into the culture medium; and
purifying the secreted polypeptide from the culture medium.

22. A method of generating a cell producing a secreted polypeptide, the method comprising:
introducing into a cell a heterologous nucleic acid encoding a secreted polypeptide;
culturing the cell under conditions that allow for synthesis of the secreted polypeptide;
contacting the cell with a compound that specifically binds to the secreted polypeptide;
detecting expression of the secreted polypeptide by binding of the compound to the secreted polypeptide on the surface of the cell; and
selecting the cell by fluorescence activated cell sorting.

23. A method of determining the presence or amount of a secreted polypeptide produced by a cell, the method comprising:
contacting a cell producing a secreted polypeptide with a compound that specifically binds to the secreted polypeptide, wherein the cell is not a B cell or a cell formed by the fusion of a B cell with another cell; and
detecting the binding of the compound to the secreted polypeptide on the surface of the cell,
to thereby determine the presence or amount of the secreted polypeptide produced by the cell.

24. The method of claim 23, wherein the cell comprises a heterologous nucleic acid encoding the secreted polypeptide.

25. A method of selecting a cell, the method comprising:
providing a cell population comprising a plurality of cells genetically engineered to contain a nucleic acid encoding a secreted polypeptide;
contacting the cell population with a compound that specifically binds to the secreted polypeptide; and
selecting a cell on the surface of which the compound is bound.
